(19) United States
(12) Patent Application Publication
(10) Pub. No.: US 2005/0228031 A1
(43) Pub. Date: Oct. 13, 2005

(54) Tyrosine kinase inhibitors

(76) Inventors: Mark T. Bilodeau, Lansdale, PA (US); Leonard Rodman, New York, NY (US)

Correspondence Address: MERCK AND CO., INC, P O BOX 2000, RAHWAY, NJ, 07065-0907, US

(21) Appl. No.: 10/823,156

(22) Filed: Apr. 13, 2004

(51) Int. Cl. A61K 31/426; C07D 277/18

(52) U.S. Cl. 514/370; 548/190

(57) ABSTRACT

The present invention relates to compounds which inhibit, regulate and/or modulate tyrosine kinase signal transduction, compositions which contain these compounds, and methods of using them to treat tyrosine kinase-dependent diseases and conditions, such as angiogenesis, cancer, tumor growth, atherosclerosis, age related macular degeneration, diabetic retinopathy, inflammatory diseases, and the like in mammals.

### BACKGROUND OF THE INVENTION

The present invention relates to compounds which inhibit, regulate and/or modulate tyrosine kinase signal transduction, compositions which contain these compounds, and methods of using them to treat tyrosine kinase-dependent diseases and conditions, such as angiogenesis, cancer, tumor growth, atherosclerosis, age related macular degeneration, diabetic retinopathy, inflammatory diseases, and the like in mammals.

Tyrosine kinases are a class of enzymes that catalyze the transfer of the terminal phosphate of adenosine triphosphate to tyrosine residues in protein substrates, as described in U.S. Pat. No. 6,245,759 B1 (hereby incorporated by reference).

Angiogenesis is characterized by excessive activity of vascular endothelial growth factor (VEGF) (as described in U.S. Pat. No. 6,245,759 B1). KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF mRNA and protein are elevated by conditions such as retinal vein occlusion in primates and decreased PO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Inhibition of KDR or FIt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

### SUMMARY OF THE INVENTION

The present invention relates to substituted aryl-aminothiazole compounds that are capable of inhibiting, modulating and/or regulating signal transduction of both receptor-type and non-receptor type tyrosine kinases.

### DETAILED DESCRIPTION OF THE INVENTION

The compounds of this invention are useful in the inhibition of kinases and are illustrated by a compound of Formula I: 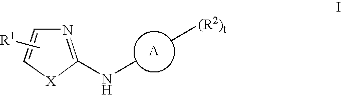
wherein 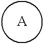
is an aryl or heterocyclyl;
X is S or O;
R is independently selected from
   1) H,
   2) unsubstituted or substituted C₁-C₁₀ alkyl,
   3) (CR₂)ₙOR⁴, and
   4) unsubstituted or substituted aryl;
R¹ is
   1) unsubstituted or substituted phenyl,
   2) CN, or
   3)—C(O)NR⁴²;
R² is independently selected from:
   1) H,
   2) CN,
   3) Halo,
   4) (CR₂)ₙOR⁴,
   5) CF₃,
   6) unsubstituted or substituted C₁-C₁₀ alkyl,
   7) unsubstituted or substituted C₃-C₁₀ cycloalkyl,
   8) unsubstituted or substituted aryl,
   9) unsubstituted or substituted aralkyl,
   10) unsubstituted or substituted heterocyclyl,
   11) unsubstituted or substituted heterocyclylalkyl,
   12) —(CR₂)ₙC(O)NR⁴₂,
   13)—(CR₂)ₙC(O)R⁴,
   14) —(CR₂)ₙR⁶C(O)R⁴₂, and
   15) —(CR₂)ₙC(O)OR⁴;
R⁴ is independently selected from:
   1) H,
   2) unsubstituted or substituted C₁-C₁₀ alkyl,
   3) unsubstituted or substituted aryl,
   4) unsubstituted or substituted aralkyl,
   5) unsubstituted or substituted heterocyclyl, and
   6) unsubstituted or substituted heterocyclylalkyl;
R⁶ is unsubstituted or substituted heterocyclyl;
n is independently 0, 1, 2, 3, 4, 5, or 6;
t is 0, 1, 2, or 3;
   or a pharmaceutically acceptable salt thereof.

In a second embodiment, the instant invention is a compound of Formula I, as described above, or a pharmaceutically acceptable salt thereof, wherein 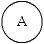
is an aryl or pyridyl;
n is independently 0, 1, 2, 3, or 4;
   and all other substituents and variables are as defined above.

In a third embodiment, the instant invention is a compound of Formula I, as described above in the first embodiment, or a pharmaceutically acceptable salt thereof, wherein 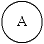
is phenyl, naphthyl, dihydroindenyl, indenyl, or pyridyl;
R is independently selected from
   1) H,
   2) unsubstituted or substituted C₁-C₁₀ alkyl, and
   3) (CR₂)ₙOR⁴;
R² is independently selected from:
   1) H,
   2) CN,
   3) Halo,
   4) (CR₂)ₙOR⁴,
   5) CF₃,
   6) unsubstituted or substituted C₁-C₁₀ alkyl,
   7) unsubstituted or substituted C₃-C₁₀ cycloalkyl,
   8) —CR₂)ₙC(O)NR⁴₂,
   9)—CR₂)ₙC(O)R⁴, and
   10) —CR₂)ₙR⁶C(O)R⁴₂;
n is independently 0, 1, 2, 3, or 4;
   and all other substituents and variables are as defined above in the first embodiment.

A specific example of the instant invention is a compound selected from:
N,5-diphenyl-1,3-thiazol-2-amine;
N-(1-naphthyl)-5-phenyl-1,3-thiazol-2-amine;
N-(3-methoxyphenyl)-5-phenyl-1,3-thiazol-2-amine;
N-[4-(benzyloxy)phenyl]-5-phenyl-1,3-thiazol-2-amine;
N-(4-methoxyphenyl)-5-phenyl-1,3-thiazol-2-amine;
4-[(5-phenyl-1,3-thiazol-2-yl)amino]benzonitrile;
N-(2-chlorophenyl)-5-phenyl-1,3-thiazol-2-amine;
N-(2,4-dimethoxyphenyl)-5-phenyl-1,3-thiazol-2-amine;
N-(3-chlorophenyl)-5-phenyl-1,3-thiazol-2-amine;
N-(4-phenoxyphenyl)-5-phenyl-1,3-thiazol-2-amine;
N-(2,5-dimethoxyphenyl)-5-phenyl-1,3-thiazol-2-amine;
N-(2,5-dichlorophenyl)-5-phenyl-1,3-thiazol-2-amine;
N-(4-chlorophenyl)-5-phenyl-1,3-thiazol-2-amine;
N-(2,6-dichlorophenyl)-5-phenyl-1,3-thiazol-2-amine;
N-(2-methoxyphenyl)-5-phenyl-1,3-thiazol-2-amine;
N-(2,4-dichlorophenyl)-5-phenyl-1,3-thiazol-2-amine;
N-(3,4-dichlorophenyl)-5-phenyl-1,3-thiazol-2-amine;
N-(4-cyclohexylphenyl)-5-phenyl-1,3-thiazol-2-amine;
5-phenyl-N-[3-(trifluoromethyl)phenyl]-1,3-thiazol-2-amine;
N-(3,5dichlorophenyl)-5-phenyl-1,3-thiazol-2-amine;
4-[(5-phenyl-1,3-thiazol-2-yl)amino]phenol;
N-(3,5-dimethylphenyl)-5-phenyl-1,3-thiazol-2-amine;
2-Anilino-1,3-thiazole-5-carbonitrile;
2-[(3,5-dimethylphenyl)amino]-1,3-thiazole-5-carbonitrile;
3-[(5-cyano-1,3-thiazol-2-yl)amino]-N,N-dimethylbenzamide;
3-[(5-cyano-1,3-thiazol-2-yl)amino]-N,N,2-trimethylbenzamide;
3-[(5-cyano-1,3-thiazol-2-yl)amino]-N,N,4-trimethylbenzamide;
4-[(5-cyano-1,3-thiazol-2-yl)amino]-N,N-dimethylbenzamide;
2-{[3-(pyrrolidin-1-ylcarbonyl)phenyl]amino}-1,3-thiazole-5-carbonitrile;
5-[(5-cyano-1,3-thiazol-2-yl)amino]-N,N′,N′-tetramethylisophthalamide;
2-{[3-(hydroxymethyl)-5-methylphenyl]amino}-1,3-thiazole-5-carbonitrile;
2-({3-[(4-Acetylpiperazin-1-yl)methyl]-5-methylphenyl}amino)-1,3-thiazole-5-carbonitrile;
2-({3-[(4-Acetylpiperazin-1-yl)methyl]phenyl}amino)-1,3-thiazole-5-carbonitrile;
Methyl 2-anilino-1,3-thiazole-5-carboxylate;
2-Anilino-1,3-thiazole-5-carboxylic acid;
2-Anilino-N-benzyl-1,3-thiazole-5-carboxamide;
2-Anilino-N,N-dimethyl-1,3-thiazole-5-carboxamide;
N-(3,5-Dimethylphenyl)-5-phenyl-1,3-oxazol-2-amine;
N-(3,5-Dimethoxyphenyl)-5-phenyl-1,3-oxazol-2-amine;
N,5-diphenyl-1,3-oxazol-2-amine;
N-(2,3-dihydro-1H-inden-5-yl)-5-phenyl-1,3-oxazol-2-amine;
N-[3,5-bis(trifluoromethyl)phenyl]-5-phenyl-1,3-oxazol-2-amine;
N-(5-phenyl-1,3-thiazol-2-yl)pyridin-3-amine;
N-(5-phenyl-1,3-thiazol-2-yl)pyridin4-amine;
   or a pharmaceutically acceptable salt thereof.

An embodiment of the instant invention are compounds selected from
N-(3,5-dimethylphenyl)-5-phenyl-1,3-thiazol-2-amine;
2-[(3,5-dimethylphenyl)amino]-1,3-thiazole-5-arbonitrile; or
2-({3-[(4-Acetylpiperazin-1-yl)methyl]-5-methylphenyl}amino)-1,3-thiazole-5-carbonitrile;
   or a pharmaceutically acceptable salt thereof.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

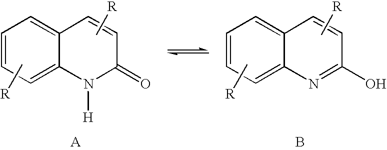

When any variable or substituent (e.g. R^{1a}, R³, n, etc.) occurs more than one time in any constituent, its definition on each occurrence is independent at every other occurrence. Also, combinations of substituents and variables are permissible only if such combinations result in stable compounds.

Lines drawn into the ring systems from substituents indicate that the indicated bond may be attached to any of the substitutable ring carbon atoms or heteroatoms, including the carbon atom or heteroatom that is the point of attachment. If the ring system is polycyclic it is intended that the bond may be attached to any of the suitable carbon atoms or heteroatoms of any ring.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrange-ment. For example, “C₁-C₁₀ alkyl” specifically includes methyl, ethyl, propyl, isopropyl, butyl, t-butyl, pentyl, hexyl, heptyl, octyl, nonyl, decyl, and so on.

“Cycloalkyl” as used herein is intended to include non-aromatic cyclic hydrocarbon groups, having the specified number of carbon atoms, which may or may not be bridged or structurally constrained. Examples of such cycloalkyls include, but are not limited to, cyclopropyl, cyclobutyl, cyclopentyl, cyclohexyl, adamantyl, cyclooctyl, cycloheptyl, tetrahydro-naphthalene, methylenecylohexyl, and the like. As used herein, examples of “C₃-C₁₀ cycloalkyl” may include, but are not limited to:

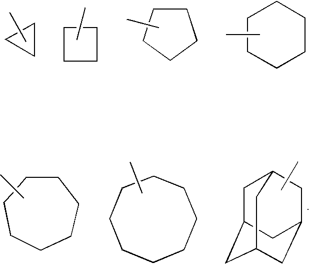

As used herein, the term “alkoxy” represents an alkyl group of indicated number of carbon atoms attached through an oxygen bridge.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 7 atoms in each ring, wherein at least one ring is aromatic. Examples of such aryl elements include phenyl, naphthyl, dihydroindenyl, tetrahydronaphthyl, indanyl, indanonyl, indenyl, biphenyl, tetralinyl, tetralonyl, fluorenonyl, phenanthryl, anthryl, acenaphthyl, tetrahydronaphthyl, and the like. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term heterocycle or heterocyclic or heterocyclyl, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. “Heterocycle” or “heterocyclyl” therefore includes the above mentioned heteroaryls, as well as dihydro and tetrathydro analogs and N-oxides thereof. Further examples of “heterocyclyl” include, but are not limited to the following: benzodioxolyl, benzofuranyl, benzofurazanyl, benzimidazolyl, benzopyranyl, benzopyrazolyl, benzotriazolyl, benzothiazolyl, benzothienyl, benzothiofuranyl, benzothiophenyl, benzothiopyranyl, benzoxazolyl, carbazolyl, carbolinyl, chromanyl, cinnolinyl, diazapinonyl, dihydrobenzodioxinyl, dihydrobenzofuranyl, dihydrobenzofuryl, dihydrobenzoimidazolyl, dihydrobenzothienyl, dihydrobenzothiopyranyl, dihydrobenzothiopyranyl sulfone, dihydrobenzothiophenyl, dihydrobenzoxazolyl, dihydrocyclopentapyridinyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxanyl, dioxolanyl, dioxidotetrahydrothienyl, dioxidothiomorpholinyl, furyl, furanyl, imidazolyl, imidazolinyl, imidazolidinyl, imidazothiazolyl, imidazopyridinyl, indazolyl, indolazinyl, indolinyl, indolyl, isobenzofuranyl, isochromanyl, isoindolyl, isoindolinyl, isoquinolinone, isoquinolyl, isothiazolyl, isothiazolidinyl, isoxazolinyl, isoxazolyl, methylenedioxybenzoyl, morpholinyl, naphthpyridinyl, oxadiazolyl, oxazolyl, oxazolinyl, oxetanyl, oxidothiomorpholinyl, oxoazepinyl, oxadiazolyl, oxodihydrophthalazinyl, oxodihydroindolyl, oxodihydrotriazolyl, oxoimidazolidinyl, oxopiperazinyl, oxopiperdinyl, oxopyrrolidinyl, oxopyrimidinyl, oxopyrrolyl, oxotriazolyl, piperidyl, piperidinyl, piperazinyl, pyranyl, pyrazinyl, pyrazolyl, pyridazinyl, pyridinonyl, pyridopyridinyl, pyridazinyl, pyridyl, pyridinyl, pyrimidinyl, pyrrolyl, pyrrolidinyl, quinazolinyl, quinolinyl, quinolyl, quinolinonyl, quinoxalinyl, tetrahydrobenzoannulenyl, tetrahydrocycloheptapyridinyl, tetrahydrofuranyl, tetrahydrofuryl, tetrahydroisoquinolinyl, tetrahydropyranyl, tetrahydroquinolinyl, tetrazolyl, tetrazolopyridyl, thiadiazolyl, thiazolyl, thiazolinyl, thienofuryl, thienyl, triazolyl, azetidinyl, 1,4-dioxanyl, hexahydroazepinyl, and the like. Attachment of a heterocyclyl substituent can occur via a carbon atom or via a heteroatom.

In an embodiment of the instant invention, heterocycle is selected from oxoazepinyl, benzimidazolyl, dioxanyl, dioxolanyl, dioxanyl, dioxidotetrahydrothienyl, oxetanyl, piperidinyl, pyrazolyl, pyridinyl, tetrahydrofuranyl, tetrahydropyranyl, imidazolyl, morpholinyl, piperidyl, piperazinyl, pyridyl, pyrrolidinyl, oxopiperidinyl, oxopyrrolidinyl, quinolinyl, tetrahydrofuryl, and N-oxides thereof.

As used herein, “aralkyl” is intended to mean an aryl moiety, as defined above, attached through a C₁-C₁₀ alkyl linker, where alkyl is defined above. Examples of aralkyls include, but are not limited to, benzyl, naphthylmethyl and phenylpropyl.

As used herein, “heterocyclylalkyl” is intended to mean a heterocyclic moiety, as defined below, attached through a C₁-C₁₀ alkyl linker, where alkyl is defined above. Examples of heterocyclylalkyls include, but are not limited to, pyridylmethyl, imidazolylethyl, pyrrolidinylmethyl, morpholinylethyl, quinolinylmethyl, imidazolylpropyl and the like.

As used herein, the terms “substituted C₁-C₁₀ alkyl” and “substituted C₁-C₆ alkoxy” are intended to include the branch or straight-chain alkyl group of the specified number of carbon atoms, wherein the carbon atoms may be substituted with 1 to 3 substituents selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆ alkyl)O(C₁-C₆ alkyl)-, (C₀-C₆ alkyl)C(O)1-2(C₀-C₆ alkyl)-, (C₀-C₆ alkyl)OC(O)NH—, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

As used herein, the terms “substituted C₃-C₁₀ cycloalkyl”, “substituted aryl”, “substituted phenyl”, “substituted heterocycle”, “substituted aralkyl” and “substituted heterocyclylalkyl” are intended to include the cyclic group containing from 1 to 3 substituents in addition to the point of attachment to the rest of the compound. Preferably, the substituents are selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂-, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-C₆ alkyl)C(O)₁₋₂(C₀-C₆ alkyl)-, (C₀-C₆ alkyl) OC(O)NH—, aryl, aralkyl, heteroaryl, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

As used herein, the phrase “substituted with at least one substituent” is intended to mean that the substituted group being referenced has from 1 to 6 substituents. Preferably, the substituted group being referenced contains from 1 to 3 substituents, in addition to the point of attachment to the rest of the compound.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Abbreviations, which may be used in the description of the chemistry and in the Examples that follow, include:

| | |
|---|---|
| Ac₂O | Acetic anhydride; |
| AcOH | Acetic acid; |
| AIBN | 2,2′-Azobisisobutyronitrile; |
| Ar | Aryl; |
| BINAP | 2,2′-Bis(diphenylphosphino)-1,1′ binaphthyl; |
| Bn | Benzyl; |
| BOC/Boc | tert-Butoxycarbonyl; |
| BSA | Bovine Serum Albumin; |
| CAN | Ceric Ammonia Nitrate; |
| CBz | Carbobenzyloxy; |
| CI | Chemical Ionization; |
| DBAD | Di-tert-butyl azodicarboxylate; |
| DBU | 1,8-Diazabicyclo[5.4.0]undec-7-ene; |
| DCC | 1,3 Dichlorohexylcarbodiimide; |
| DCE | 1,2-Dichloroethane; |
| DCM | Dichloromethane; |
| DIEA | N,N-Diisopropylethylamine; |
| DMAP | 4-Dimethylaminopyridine; |
| DMA | Dimethylacetamide; |
| DME | 1,2-Dimethoxyethane; |
| DMF | N,N-Dimethylformamide; |
| DMSO | Methyl sulfoxide; |
| DPPA | Diphenylphosphoryl azide; |
| DTT | Dithiothreitol; |
| EDC | 1-(3-Dimethylaminopropyl)-3-ethyl-carbodiimide- |
| | hydrochloride; |
| EDTA | Ethylenediaminetetraacetic acid; |
| ELSD | Evaporative Light Scattering Detector; |
| ES | Electrospray; |
| ESI | Electrospray ionization; |
| Et₂O | Diethyl ether; |
| Et₃N | Triethylamine; |
| EtOAc | Ethyl acetate; |
| EtOH | Ethanol; |
| FAB | Fast atom bombardment; |
| HEPES | 4-(2-Hydroxyethyl)-1-piperazineethanesulfonic acid; |
| HMPA | Hexamethylphosphoramide; |
| HOAc | Acetic acid; |
| HOBT | 1-Hydroxybenzotriazole hydrate; |
| HOOBT | 3-Hydroxy-1,2,2-benzotriazin-4(3H)-one; |
| HPLC | High-performance liquid chromatography; |
| HRMS | High Resolution Mass Spectroscopy; |
| KOtBu | Potassium tert-butoxide; |
| LAH | Lithium aluminum hydride; |
| LCMS | Liquid Chromatography Mass Spectroscopy; |
| MCPBA | m-Chloroperoxybenzoic acid; |
| Me | Methyl; |
| MeOH | Methanol; |
| Ms | Methanesulfonyl; |
| MS | Mass Spectroscopy; |
| MsCl | Methanesulfonyl chloride; |
| n-Bu | n-butyl; |
| n-Bu₃P | Tri-n-butylphosphine; |
| NaHMDS | Sodium bis(trimethylsilyl)amide; |
| NBS | N-Bromosuccinimide; |
| NMM | N-methylmorpholine; |
| NMR | Nuclear Magnetic Resonance; |
| Pd(PPh₃)₄ | Palladium tetrakis(triphenylphosphine); |
| Pd₂(dba)₃ | Tris(dibenzylideneacetone)dipalladium (0); |
| Ph | Phenyl; |
| PMSF | □-Toluenesulfonyl fluoride; |
| PS-DCC | Polystyrene dicyclohexylcarbodiimide; |
| PS-DMAP | Polystyrene dimethylaminopyridine; |
| PS-NMM | Polystyrene N-methylmorpholine; |
| Py or pyr | Pyridine; |
| PYBOP | Benzotriazol-1-yloxytripyrrolidinophosphonium |
| (or PyBOP) | hexafluorophosphate; |
| RPLC | Reverse Phase Liquid Chromatography; |
| RT | Room Temperature; |
| SCX SPE | Strong Cation Exchange Solid Phase Extraction; |
| t-Bu | tert-Butyl; |
| TBAF | Tetrabutylammonium fluoride; |
| TBSCl | tert-Butyldimethylsilyl chloride; |
| TFA | Trifluoroacetic acid; |
| THF | Tetrahydrofuran; |
| TIPS | Triisopropylsilyl; |
| TLC | Thin layer chromatography; |
| TMS | Tetramethylsilane; and |
| Tr | Trityl. |

The compounds of this invention may be prepared by employing reactions as shown in the following schemes, in addition to other standard manipulations that are known in the literature or exemplified in the experimental procedures. These schemes, therefore, are not limited by the compounds listed or by any particular substituents employed for illustrative purposes. Substituent numbering as shown in the schemes does not necessarily correlate to that used in the claims.

### Schemes

The target thiazoles A-3 can be arrived at by reacting the appropriate thiourea A-1 with a bromo acetal A-2, as shown in Scheme A.

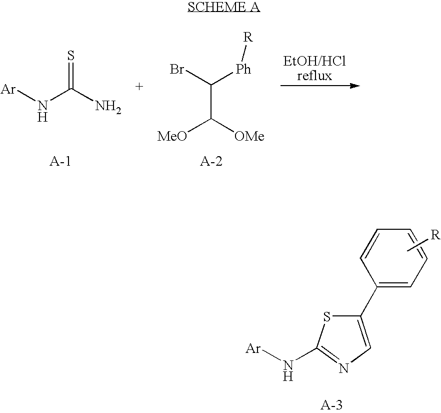

Alternatively, the N—C bond-forming protocol illustrated in Scheme B can be used to obtain thiazoles and oxazoles of Formula B-3.

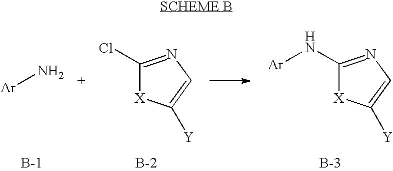

Scheme C illustrates the synthesis of cyano-thiazole compounds of Formula C-3.

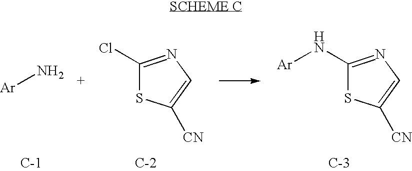

### Utilities

The compounds of the present invention are inhibitors of tyrosine kinase and are therefore useful to treat or prevent tyrosine kinase-dependent diseases or conditions in mammals.

“Tyrosine kinase-dependent diseases or conditions” refers to pathologic conditions that depend on the activity of one or more tyrosine kinases. Tyrosine kinases either directly or indirectly participate in the signal transduction pathways of a variety of cellular activities including proliferation, adhesion and migration, and differentiation. Diseases associated with tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like). In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

The present invention encompasses a method of treating or preventing cancer in a mammal in need of such treatment which is comprised of administering to said mammal a therapeutically effective amount of a claimed compound. Preferred cancers for treatment are selected from cancers of the brain, genitourinary tract, lymphatic system, stomach, larynx and lung. Another set of preferred forms of cancer are histiocytic lymphoma, lung adenocarcinoma, small cell lung cancers, pancreatic cancer, glioblastomas and breast carcinoma. The utility of angiogenesis inhibitors in the treatment of cancer is known in the literature, see J. Rak et al. Cancer Research, 55:45754580, 1995, for example. The role of angiogenesis in cancer has been shown in numerous types of cancer and tissues: breast carcinoma (G. Gasparini and A. L. harris, J. Clin. Oncol., 1995, 13:765-782; M. Toi et al., Japan. J. Cancer Res., 1994, 85:1045-1049); bladder carcinomas (A. J. Dickinson et al., Br. J. Urol., 1994, 74:762-766); colon carcinomas (L. M. Ellis et al., Surgery, 1996, 120(5):871-878); and oral cavity tumors (J. K. Williams et al., Am. J. Surg., 1994, 168:373-380).

Tumors which have undergone neovascularization show an increased potential for metastasis. VEGF released from cancer cells enhances metastasis possibly by increasing extravasation at points of adhesion to vascular endothelium. (A. Amirkhosravi et al., Platelets, 10:285-292 (1999)). In fact, angiogenesis is essential for tumor growth and metastasis. (S. P. Cunningham, et al., Can. Research, 61: 3206-3211 (2001)). The angiogenesis inhibitors disclosed in the present application are therefore also useful to prevent or decrease tumor cell metastasis. Such a use is also contemplated to be within the scope of the present invention.

Further included within the scope of the invention is a method of treating or preventing a disease in which angiogenesis is implicated, which is comprised of administering to a mammal in need of such treatment a therapeutically effective amount of a compound of the present invention. Ocular neovascular diseases are an example of conditions where much of the resulting tissue damage can be attributed to aberrant infiltration of blood vessels in the eye (see WO 00/30651, published 2 Jun. 2000). The undesireable infiltration can be triggered by ischemic retinopathy, such as that resulting from diabetic retinopathy, retinopathy of prematurity, retinal vein occlusions, etc., or by degenerative diseases, such as the choroidal neovascularization obeserved in age-related macular degeneration. Inhibiting the growth of blood vessels by administration of the present compounds would therefore prevent the infiltration of blood vessels and prevent or treat diseases where angiogenesis is implicated, such as ocular diseases like retinal vascularization, diabetic retinopathy, age-related macular degeneration, and the like.

Also included within the scope of the present invention is a method of treating or preventing inflammatory diseases which comprises administering to a mammal in need of such treatment a therapeutically effective amount of a compound of Formual I. Examples of such inflammatory diseases are rheumatoid arthritis, psoriasis, contact dermatitis, delayed hypersensitivity reactions, and the like. (A. Giatromanolaki et al., J. Pathol. 2001; 194:101-108.) For the role of VEGF in skin angiogenesis, see Michael Detmar, J. Dernatological Sci., 24 Suppl. 1, S78-S84 (2000).

Also included within the scope of the present invention is a method of treating or preventing bone associated pathologies selected from osteosarcoma, osteoarthritis, and rickets, also known as oncogenic osteomalacia. (Hasegawa et al., Skeletal Radiol., 28, pp. 41-45, 1999; Gerber et al., Nature Medicine, Vol. 5, No. 6, pp. 623-628, June 1999.) And since VEGF directly promotes osteoclastic bone resorption through KDR/Flk-1 expressed in mature osteoclasts (FEBS Let. 473:161-164 (2000); Endocrinology, 141:1667 (2000)), the instant compounds are also useful to treat and prevent conditions related to bone resorption, such as osteoporosis and Paget's disease.

A method of treating or preventing preeclampsia is also within the scope of this invention which comprises administering a therapeutically effective amount of a compound of the present invention. Studies have shown that the action of VEGF on the Flt-1 receptor is pivotal in the pathogenesis of preeclampsia. (Laboratory Investigation 79:1101-1111 (September 1999).) Vessels of pregnant women incubated with VEGF exhibit a reduction in endothelium-dependent relaxation similar to that induced by plasma from women with preeclampsia. In the presence of an anti-Flt-1 receptor antibody, however, neither VEGF nor plasma from women with preeclampsia reduced the endothelium-dependent relaxation. Therefore the claimed compounds serve to treat preeclampsia via their action on the tyrosine kinase domain of the Flt-1 receptor.

Also within the scope of the invention is a method of reducing or preventing tissue damage following a cerebral ischemic event which comprises administering a therapeutically effective amount of a compound of the present invention. The claimed compounds can also be used to reduce or prevent tissue damage which occurs after cerebral ischemic events, such as stroke, by reducing cerebral edema, tissue damage, and reperfusion injury following ischemia. (Drug News Perspect 11:265-270 (1998); J. Clin. Invest. 104:1613-1620 (1999); Nature Med 7:222-227 (2001)).

The instant compounds can also be used to prevent or treat tissue damage during bacterial meningitis, such as tuberculous meningitis. Matsuyama et al., J. Neurol. Sci. 186:75-79 (2001)). The instant invention therefore encompasses a method of treating or preventing tissue damage due to bacterial meningitis which comprises administering a therapeutically effective amount of a claimed compound. Studies have shown that VEGF is secreted by inflammatory cells during bacterial meningitis and that VEGF contributes to blood-brain barrier disruption. (van der Flier et al., J. Infectious Diseases, 183:149-153 (2001)). The claimed compounds can inhibit VEGF-induced vascular permeability and therefore serve to prevent or treat blood-brain barrier disruption associated with bacterial meningitis.

The present invention further encompasses a method to treat or prevent endometrioses comprised of administering a therapeutically effective amount of a claimed compound. An increase in VEGF expression and angiogenesis is associated with the progression of endometriosis (Stephen K. Smith, Trends in Endocrinology & Metabolism, Vol. 12, No. 4, May/June 2001). Inhibition of VEGF by the current compounds would therefore inhibit angiogenesis and treat endometriosis.

A further embodiment of the present invention is a method of treating acute myeloid leukemia (AML) which comprises administering a therapeutically effective amount of a claimed compound. Activation of FLT3 on leukemic cells by FLT3 ligand leads to receptor dimerization and signal transduction in pathways that promote cell growth and inhibit apoptosis (Blood, Vol. 98, No. 3, pp. 885-887 (2001)). The present compounds are therefore useful to treat AML via inhibition of the tyrosine kinase domain of Flt-3.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

For oral use of a chemotherapeutic compound according to this invention, the selected compound may be administered, for example, in the form of tablets or capsules, or as an aqueous solution or suspension. In the case of tablets for oral use, carriers which are commonly used include lactose and cornstarch, and lubricating agents, such as magnesium stearate, are commonly added. For oral administration in capsule form, useful diluents include lactose and dried cornstarch. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The instant compounds are also useful in combination with known therapeutic agents and anti-cancer agents, as described in U.S. Pat. No. 6,245,759 B1 (columns 14-19) and PCT Publ. No. WO 01/29025 (pages 3341) (both of which are incorporated by reference). Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers.

A person of ordinary skill in the art would be able to discern which combinations of agents would be useful based on the particular characteristics of the drugs and the cancer involved. Such anti-cancer agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, and agents that interfere with cell cycle checkpoints. The instant compounds are particularly useful when co-administered with radiation therapy. The synergistic effects of inhibiting VEGF in combination with radiation therapy have been described in the art (see WO 00/61186).

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2dimethyl-1-oxopropoxy4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ELX23-7553, trans-N-(4′-hydroxyphenyl) retinamide, and N4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, antimetabolites; biological response modifiers; tumor necrosis factors, intercalators, hormonal/anti-hormonal therapeutic agents, haematopoietic growth factors, monoclonal antibody targeted therapeutic agents, topoisomerase inhibitors, proteosome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX 100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3deamino-3-aziridinyl-4-methylsulphonyl-daunorubicin (see WO 00/50032).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy4-methyl-1H,12H-benzo[de]pyrano[3′,4′:b,7]-indolizino[1,2b]quinoline-10,13(9H, 15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen4-ylmethyl]formamide, N-(2-(dimethylamino)ethyl)acridine4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768 and WO 01/98278, and pending U.S. Ser. Nos. 60/338,779 (filed Dec. 6, 2001), 60/338,344 (filed Dec. 6, 2001), 60/338,383 (filed Dec. 6, 2001), 60/338,380 (filed Dec. 6, 2001), 60/338,379 (filed Dec. 6, 2001) and 60/344,453 (filed Nov. 7, 2001). In an embodiment inhibitors of mitotic kinesins include, but are not limited to inhibitors of KSP, inhibitors of MKLP1, inhibitors of CENP-E, inhibitors of MCAK, inhibitors of aurora kinase and inhibitors of Rab6-KIFL.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine, 3-aminopyridine-2-carboxaldehyde thiosemicarbazone and trastuzumab. Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

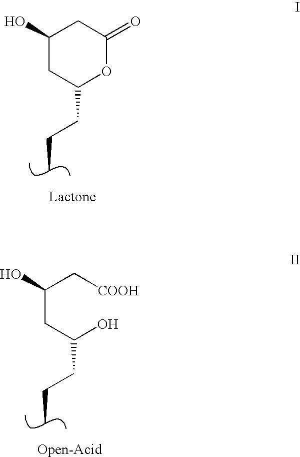

In HMG-CoA reductase inhibitors where an open-acid form can exist, salt and ester forms may be formed from the open-acid, and all such forms are included within the meaning of the term “HMG-CoA reductase inhibitor” as used herein. In an embodiment, the HMG-CoA reductase inhibitor is selected from lovastatin and simvastatin, and in a further embodiment, simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPI ase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]4-(3-chlorophenyl)-1-methyl-2(1H)-uinolinone, (−)-6-[amino(4-chlorophenyl)(1-methyl-1H-imidazol-5-yl)methyl]4-(3-chlorophenyl)-1-methyl-2(1H)uinolinone, (+)-6-[amino(4-chlorophenyl)(1-methyl-1H-imidazol-5-yl) methyl] 4-(3-chlorophenyl)-1-methyl-2(11H)-uinolinone, 5(S)-n-butyl-1-(2,3-dimethylphenyl)₄-[1-(4-cyanobenzyl)-5-imidazolylmethyl]-2-piperazinone, (S)-1-(3-chlorophenyl) 4-[1-(4-cyanobenzyl)-5-imidazolylmethyl]-5-[2-(ethanesulfonyl) methyl)-2-piperazinone, 5(S)-n-Butyl-1-(2-methylphenyl)₄-[1-(4-cyanobenzyl)-5-imidazolylmethyl]-2-piperazinone, 1-(3-chlorophenyl) 4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′ ]bipyridin-5′-ylmethyl]-3H-imidazol-4-ylmethyl}benzonitrile, 4-{3-[4-(2-oxo-2H-[1,2′]bipyridin-5′-ylmethyl]-3H-imidazol-4-ylmethyl}benzonitrile, 4-[3-(2-oxo-1-phenyl-1,2-dihydropyridin4-ylmethyl)-3H-imidazol-4-ylmethyl}benzonitrile, 18,19-dihydro-19-oxo-5H,17H-6,10:12,16-dimetheno-1H-imidazo[4,3-c][1,11,4]dioxaazacyclo-nonadecine-9-carbonitrile, (+)-19,20-dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacycloeicosine-9-carbonitrile, and (±)-19,20-dihydro-3-methyl-19-oxo-5H-18,21-ethano-12,14-etheno-6,10-metheno-22H-benzo [d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

Examples of HIV protease inhibitors include amprenavir, abacavir, CGP-73547, CGP-61755, DMP450, indinavir, nelfinavir, tipranavir, ritonavir, saquinavir, ABT-378, AG 1776, and BMS-232,632. Examples of reverse transcriptase inhibitors include delaviridine, efavirenz, GS-840, HB Y097, lamivudine, nevirapine, AZT, 3TC, ddC, and ddI.

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR 1) and Flk-1/KDR (VEGFR2), inhibitors of epidermal-derived, fibroblast-derived, or platelet derived growth factors, MMP (matrix metalloprotease) inhibitors, integrin blockers, interferon-ax, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflanmmatories (such as corticosteroids, mineralocorticoids, dexamethasone, prednisone, prednisolone, methylpred, betamethasone), carboxyamidotriazole, combretastatin A4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

The instant compounds are also useful, alone or in combination with platelet fibrinogen receptor (GP IIb/IIIa) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Ainirkhosravi, Platelets 10, 285-292, 1999). Therefore, the present compounds can serve to inhibit metastasis, alone or in combination with GP IIb/IIIa) antagonists. Examples of other fibrinogen receptor antagonists include abciximab, eptifibatide, sibrafiban, lamifiban, lotrafiban, cromofiban, and CT50352.

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140 and WO 02/083138), inhibitors of Raf kinase (for example BAY43-9006), inhibitors of MEK (for example C₁₋₁₀₄₀ and PD-098059), inhibitors of mTOR (for example Wyeth CCI-779), and inhibitors of PI3K (for example LY294002).

As described above, the combinations with NSAID's are directed to the use of NSAID's which are potent COX-2 inhibiting agents. For purposes of this specification an NSAID is potent if it possess an IC₅₀ for the inhibition of COX-2 of lltM or less as measured by cell or mnicrosomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference.

Inhibitors of COX-2 that are particularly useful in the instant method of treatment are:
3-phenyl-4-(4-(methylsulfonyl)phenyl)-2-(5H)-furanone; and 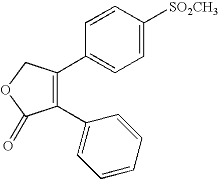
5-chloro-3-(4-methylsulfonyl)phenyl-2-(2-methyl-5-pyridinyl)pyridine; 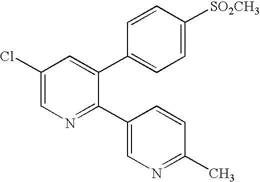
   or a pharmaceutically acceptable salt thereof.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following: 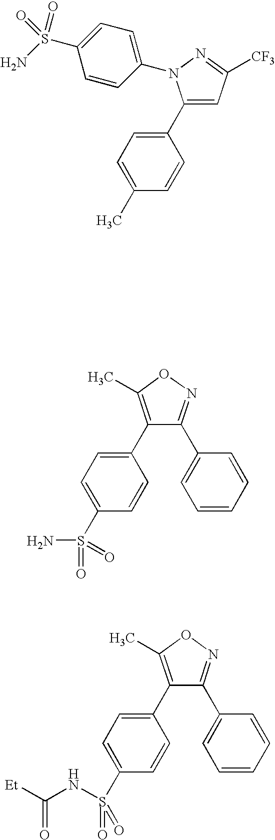
or a pharmaceutically acceptable salt thereof.

Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostatin, ukrain, ranpirnase, IM862, 5-methoxy4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM 101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the □ᵥ□₃ integrin, to compounds which selectively antagonize, inhibit or counteract binding of a physiological ligand to the □ᵥ□₅ integrin, to compounds which antagonize, inhibit or counteract binding of a physiological ligand to both the □ᵥ□₃ integrin and the □ᵥ□₅ integrin, and to compounds which antagonize, inhibit or counteract the activity of the particular integrin(s) expressed on capillary endothelial cells. The term also refers to antagonists of the □ᵥ□₆, □ᵥ□₈, □₁□₁, □₂□₁, □₅□₁, □₆□₁ and □₆□₄ integrins. The term also refers to antagonists of any combination of □ᵥ□₃, □ᵥ□₅, □ᵥ□₆, □ᵥ□₈, □₁□₁, □₂□₁, □₅□₁, □₆□₁ and □₆□₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-1 7-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg: 3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J Immunol 2000;164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. For the treatment or prevention of emesis that may result upon administration of the instant compounds, conjunctive therapy with an anti-emesis agent selected from a neurokinin-1 receptor antagonist, a 5HT3 receptor antagonist and a corticosteroid is preferred.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)₄-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

Thus, the scope of the instant invention encompasses the use of the instantly claimed compounds in combination with a second compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HUV protease inhibitor,
9) a reverse transcriptase inhibitor,
10) an angiogenesis inhibitor,
11) a PPAR-y agonists,
12) a PPAR-8 agonists,
13) an inhibitor of inherent multidrug resistance,
14) an anti-emetic agent,
15) an agent useful in the treatment of anemia,
16) an agent useful in the treatment of neutropenia,
17) an immunologic-enhancing drug,
18) an inhibitor of cell proliferation and survival signaling, and
19) an agent that interferes with a cell cycle checkpoint.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-cc, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from:
1) an estrogen receptor modulator,
2) an androgen receptor modulator,
3) a retinoid receptor modulator,
4) a cytotoxic agent,
5) an antiproliferative agent,
6) a prenyl-protein transferase inhibitor,
7) an HMG-CoA reductase inhibitor,
8) an Hw protease inhibitor,
9) a reverse transcriptase inhibitor,
10) an angiogenesis inhibitor,
11) PPAR-γ agonists,
12) PPAR-δ agonists,
13) an inhibitor of inherent multidrug resistance,
14) an anti-emetic agent,
15) an agent useful in the treatment of anemia,
16) an agent useful in the treatment of neutropenia,
17) an immunologic-enhancing drug,
18) an inhibitor of cell proliferation and survival signaling, and
19) an agent that interferes with a cell cycle checkpoint.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from:
1) an estrogen receptor modulator,
2) an androgen receptor modulator,
3) a retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor,
10) an angiogenesis inhibitor,
11) a PPAR-γ agonist,
12) a PPAR-δ agonists,
13) an inhibitor of cell proliferation and survival signaling, and
14) an agent that interferes with a cell cycle checkpoint.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The present invention also encompasses a pharmaceutical composition useful in the treatment of cancer, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

These and other aspects of the invention will be apparent from the teachings contained herein.

### Assays

The compounds of the instant invention described in the Examples were tested by the assays described below and were found to have kinase inhibitory activity. Other assays are known in the literature and could be readily performed by those of skill in the art (see, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413427; Nicosia et al., In Vitro 18:538-549).

I. VEGF Receptor Kinase Assay

VEGF receptor kinase activity is measured by incorporation of radio-labeled phosphate into polyglutamic acid, tyrosine, 4:1 (pEY) substrate. The phosphorylated pEY product is trapped onto a filter membrane and the incorporation of radio-labeled phosphate quantified by scintillation counting.

### Materials

VEGF Receptor Kinase

The intracellular tyrosine kinase domains of human KDR (Terman, B. I. et al. Oncogene (1991) vol. 6, pp. 1677-1683.) and Flt-1 (Shibuya, M. et al. Oncogene (1990) vol. 5, pp. 519-524) were cloned as glutathione S-transferase (GST) gene fusion proteins. This was accomplished by cloning the cytoplasmic domain of the KDR kinase as an in frame fusion at the carboxy terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

The other materials used and their compositions were as follows: Lysis buffer: 50 mM Tris pH 7.4, 0.5 M NaCl, 5 mM DTT, 1 mM EDTA, 0.5% triton X-100, 10% glycerol, 10 mg/mL of each leupeptin, pepstatin and aprotinin and 1 mM phenylmethylsulfonyl fluoride (all Sigma).
Wash buffer: 50 mM Tris pH 7.4,0.5 M NaCl, 5 mM DTT, 1 mM EDTA, 0.05% triton X-100, 10% glycerol, 10 mg/mL of each leupeptin, pepstatin and aprotinin and 1 mM phenylmethylsulfonyl fluoride.
Dialysis buffer: 50 mM Tris pH 7.4, 0.5 M NaCl, 5 mM DTT, 1 mM EDTA, 0.05% triton X-100, 50% glycerol, 10 mg/mL of each leupeptin, pepstatin and aprotinin and 1 mM phenylmethylsuflonyl fluoride.
10× reaction buffer: 200 mM Tris, pH 7.4, 1.0 M NaCl, 50 mM MnCl₂, 10 mM DTT and 5 mg/mL bovine serum albumin (Sigma).
Enzyme dilution buffer: 50 mM Tris, pH 7.4, 0.1 M NaCl, 1 mM DTT, 10% glycerol, 100 mg/mL BSA.
10× Substrate: 750 μg/mL poly (glutamic acid, tyrosine; 4:1) (Sigma).
Stop solution: 30% trichloroacetic acid, 0.2 M sodium pyrophosphate (both Fisher).
Wash solution: 15% trichloroacetic acid, 0.2 M sodium pyrophosphate.
Filter plates: Millipore #MAFC NOB, GF/C glass fiber 96 well plate.

### Method

A. Protein Purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR protein was eluted with wash buffer/10 mM reduced glutathione (Sigma) and dialyzed against dialysis buffer.

B. VEGF Receptor Kinase Assay

1. Add 5 μl of inhibitor or control to the assay in 50% DMSO.

2. Add 35 μl of reaction mix containing 5 μL of 10× reaction buffer, 5 μl 25 mM ATP/10 μCi [³³P]ATP (Amersham), and 5 μl 10× substrate.

3. Start the reaction by the addition of 10 μl of KDR (25 nM) in enzyme dilution buffer.

4. Mix and incubate at room temperature for 15 minutes.

5. Stop by the addition of 50 μl stop solution.

6. Incubate for 15 minutes at 4° C.

7. Transfer a 90 μl aliquot to filter plate.

8. Aspirate and wash 3 times with wash solution.

9. Add 30 μl of scintillation cocktail, seal plate and count in a Wallac Microbeta scintillation counter.

II. Human Umbilical Vein Endothelial Cell Mitogenesis Assay

Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H]thymidine into cellular DNA.

### Materials

HUVECs: HUVECs frozen as primary culture isolates are obtained from Clonetics Corp. Cells are maintained in Endothelial Growth Medium (EGM; Clonetics) and are used for mitogenic assays described in passages 3-7 below.
Culture Plates: NUNCLON 96-well polystyrene tissue culture plates (NUNC #167008).
Assay Medium: Dulbecco's modification of Eagle's medium containing 1 g/mL glucose (low-glucose DMEM; Mediatech) plus 10% (v/v) fetal bovine serum (Clonetics).
Test Compounds: Working stocks of test compounds are diluted serially in 100% dimethylsulfoxide (DMSO) to 400-fold greater than their desired final concentrations. Final dilutions to 1× concentration are made directly into Assay Medium immediately prior to addition to cells.
10× Growth Factors: Solutions of human VEGF165 (500 ng/mL; R&D Systems) and bFGF (10 ng/mL; R&D Systems) are prepared in Assay Medium.
10× [³H]Thymidine: [Methyl-3H]thymidine (20 Ci/mmol; Dupont-NEN) is diluted to 80 μCi/mL in low-glucose DMEM.
Cell Wash Medium: Hank's balanced salt solution (Mediatech) containing 1 mg/mL bovine serum albumin (Boehringer-Mannheim).
Cell Lysis Solution: 1 N NaOH, 2% (w/v) Na₂CO₃.

### Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 μL/well of either Assay Medium, 10×VEGF solution or 10×bFGF solution. Cells are then incubated at 37° C. and 5% CO₂.

4. After 24 hours in the presence of growth factors, 10× [³H]thymidine (10 WLlwell) is added.

5. Three days after addition of [³H]thymidine, medium is removed by aspiration, and cells are washed twice with Cell Wash Medium (400 μL/well followed by 200 giuwell). The washed, adherent cells are then solubilized by addition of Cell Lysis Solution (100 pliwell) and warming to 37° C. for 30 minutes. Cell lysates are transferred to 7-mL glass scintillation vials containing 150 μL of water. Scintillation cocktail (5 mL/vial) is added, and cell-associated radioactivity is determined by liquid scintillation spectroscopy.

Based upon the foregoing assays the compounds of the present invention are inhibitors of VEGF and thus are useful for the inhibition of angiogenesis, such as in the treatment of ocular disease, e.g., diabetic retinopathy and in the treatment of cancers, e.g., solid tumors. The instant compounds inhibit VEGF-stimulated mitogenesis of human vascular endothelial cells in culture with IC₅₀ values between 0.01-5.0 μM. These compounds may also show selectivity over related tyrosine kinases (e.g., FGFR1 and the Src family; for relationship between Src kinases and VEGFR kinases, see Eliceiri et al., Molecular Cell, Vol. 4, pp. 915-924, December 1999).

III. FLT-1 Kinase Assay

Fit-1 was expressed as a GST fusion to the Flt-1 kinase domain and was expressed in baculovirus/insect cells. The following protocol was employed to assay compounds for Flt-1 kinase inhibitory activity:
1. Inhibitors were diluted to account for the final dilution in the assay, 1:20.
2. The appropriate amount of reaction mix was prepared at room temperature:
   10× Buffer (20 mM Tris pH 7.4/0.1 M NaCl/1 mM DTT final)
   0.1M MnCl₂ (5 mM final)
   pEY substrate (75 μg/mL)
   ATP/[³³P]ATP (2.5 μM/1 μCi final)
   BSA (500 μg/mL final).
3. 5 μL of the diluted inhibitor was added to the reaction mix. (Final volume of 5 μL in 50% DMSO). To the positive control wells, blank DMSO (50%) was added.
4. 35 μL of the reaction mix was added to each well of a 96 well plate.
5. Enzyme was diluted into enzyme dilution buffer (kept at 4° C.).
6. 10 μL of the diluted enzyme was added to each well and mix (5 nM final). To the negative control wells, 10 μL 0.5 M EDTA was added per well instead (final 100 mM).
7. Incubation was then carried out at room temperature for 30 minutes.
8. Stopped by the addition of an equal volume (50 μL) of 30% TCA/0.1M Na pyrophosphate.
9. Incubation was then carried out for 15 minutes to allow precipitation.
10. Transfered to Millipore filter plate.
11. Washed 3× with 15% TCA/0.1M Na pyrophosphate (125 μL per wash).
12. Allowed to dry under vacuum for 2-3 minutes.
13. Dryed in hood for −20 minutes.
14. Assembled Wallac Millipore adapter and added 50 μL of scintillant to each well and counted.
   IV. FLT-3 Kinase Assay

Flt-3 was expressed as a GST fusion to the Flt-3 kinase domain, and was expressed in baculovirus/insect cells. The following protocol was employed to assay compounds for Flt-3 kinase inhibitory activity:
1. Dilute inhibitors (account for the final dilution into the assay, 1:20)
2. Prepare the appropriate amount of reaction mix at room temperature.
   10× Buffer (20 mM Tris pH 7.4/0.1 M NaCt/1 mM DTT final)
   0.1M MnCl₂ (5 mM final)
   pEY substrate (75 g/mL)
   ATP/[³³P]ATP (0.5 μM/L μCi final)
   BSA (500 μg/mL final)
3. Add 5 μL of the diluted inhibitor to the reaction mix. (Final volume of 5 μL in 50% DMSO). Positive control wells—add blank DMSO (50%).
4. Add 35 μL of the reaction mix to each well of a 96 well plate.
5. Dilute enzyme into enzyme dilution buffer (keep at 4° C.).
6. Add 10 μL of the diluted enzyme to each well and mix (5-10 nM final). Negative control wells—add 10 μL 0.5 M EDTA per well instead (final 100 mM)
7. Incubate at room temperature for 60 minutes.
8. Stop by the addition of an equal volume (50 μL) of 30% TCA/0.1M Na pyrophosphate.
9. Incubate for 15 minutes to allow precipitation.
10. Transfer to Millipore filter plate.
11. Wash 3× with 15% TCA/0.1M Na pyrophosphate (125 μL per wash).
12. Allow to dry under vacuum for 2-3 minutes.
13. Dry in hood for −20 minutes.
14. Assemble Wallac Millipore adapter and add 50 μL of scintillant to each well and count.

### Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 giuwell of either Assay Medium, 10×VEGF solution or 10×bFGF solution. Cells are then incubated at 37° C. and 5% CO₂.

4. After 24 hours in the presence of growth factors, 10×[³H]thymidine (10 μL/well) is added.

5. Three days after addition of [³Hlthymidine, medium is removed by aspiration, and cells are washed twice with Cell Wash Medium (400 SLuwell followed by 200 guwell). The washed, adherent cells are then solubilized by addition of Cell Lysis Solution (100 μL/well) and warming to 37° C. for 30 minutes. Cell lysates are transferred to 7-mL glass scintillation vials containing 150 μL of water. Scintillation cocktail (5 mL/vial) is added, and cell-associated radioactivity is determined by liquid scintillation spectroscopy.

Based upon the foregoing assays the compounds of Formula I are inhibitors of VEGF and thus are useful for the inhibition of angiogenesis, such as in the treatment of ocular disease, e.g., diabetic retinopathy and in the treatment of cancers, e.g., solid tumors. The instant compounds inhibit VEGF-stimulated mitogenesis of human vascular endothelial cells in culture with IC50 values between 0.001-5.0 μM. These compounds also show selectivity over related tyrosine kinases (e.g., FGFR1 and the Src family; for relationship between Src kinases and VEGFR kinases, see Eliceiri et al., Molecular Cell, Vol. 4, pp. 915-924, December 1999).

### EXAMPLES

Examples provided are intended to assist in a further understanding of the invention. Particular materials employed, species and conditions are intended to be further illustrative of the invention and not limiting of the reasonable scope thereof.

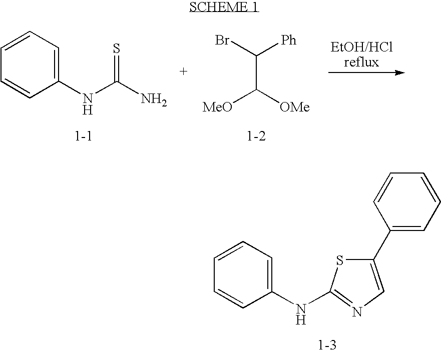

(1-Bromo-2,2-dimethoxy-ethyl)-benzene 1-2 (Bellesia, F.; Boni, M.; Ghelfi, F.; Pagnoni, U. M.; Gazz. Chim. Ital. 1993, 123, 629-632, 1.2 equiv) was dissolved to a 0.15 M solution in EtOH and was added to the appropriate thiourea (1 equiv). Aqueous hydrochloric acid (18 equiv, 12 M) was added and the reactions were heated overnight at 90° C. The reactions were concentrated in vacuo. Samples were then purified by preparative reverse phase chromatography. All other members of the table were made in the same fashion.

| | | | MS |
|---|---|---|---|
| No. | Structure | Name | (M + H) |
| 1-3 | 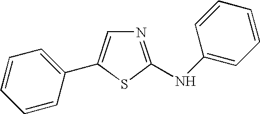 | N,5-diphenyl-1,3-thiazol-2- amine | 253.08 |
| 1-4 | 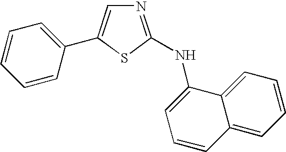 | N-(1-naphthyl)-5-phenyl- 1,3-thiazol-2-amine | 303.1 |
| 1-5 | 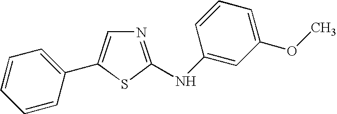 | N-(3-methoxyphenyl)-5- phenyl-1,3-thiazol-2-amine | 283.09 |
| 1-6 | 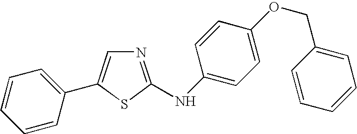 | N-[4-(benzyloxy)phenyl]- 5-phenyl-1,3-thiazol-2- amine | 359.12 |
| 1-7 | 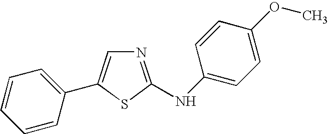 | N-(4-methoxyphenyl)-5- phenyl-1,3-thiazol-2-amine | 283.09 |
| 1-8 | 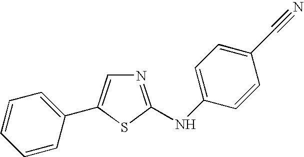 | 4-[(5-phenyl-1,3-thiazol-2- yl)amino]benzonitrile | 278.08 |
| 1-9 | 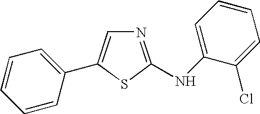 | N-(2-chlorophenyl)-5- phenyl-1,3-thiazol-2-amine | 287.04 |
| 1-10 | 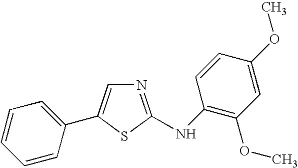 | N-(2,4-dimethoxyphenyl)- 5-phenyl-1,3-thiazol-2- amine | 313.10 |
| 1-11 | 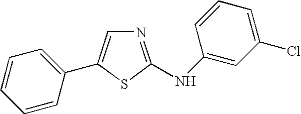 | N-(3-chlorophenyl)-5- phenyl-1,3-thiazol-2-amine | 287.04 |
| 1-12 | 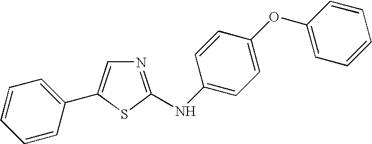 | (4-phenoxyphenyl)-5- phenyl-1,3-thiazol-2-amine | 345.11 |
| 1-13 | 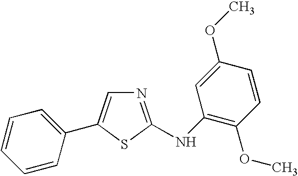 | N-(2,5-dimethoxyphenyl)- 5-phenyl-1,3-thiazol-2- amine | 313.10 |
| 1-14 | 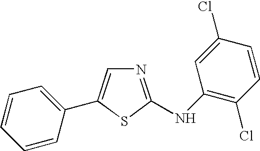 | N-(2,5-dichlorophenyl)-5- phenyl-1,3-thiazol-2-amine | 321.00 |
| 1-15 | 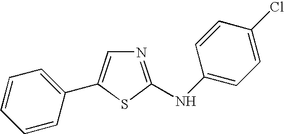 | N-(4-chlorophenyl)-5- phenyl-1,3-thiazol-2-amine | 287.04 |
| 1-16 | 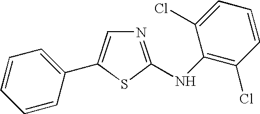 | N-(2,6-dichlorophenyl)-5- phenyl-1,3-thiazol-2-amine | 321.00 |
| 1-17 | 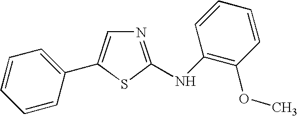 | N-(2-methoxyphenyl)-5- phenyl-1,3-thiazol-2-amine | 283.09 |
| 1-18 | 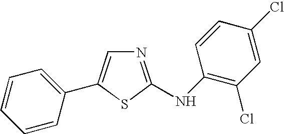 | N-(2,4-dichlorophenyl)-5- phenyl-1,3-thiazol-2-amine | 321.00 |
| 1-19 | 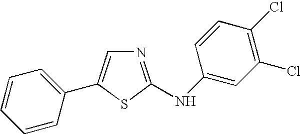 | N-(3,4-dichlorophenyl)-5- phenyl-1,3-thiazol-2-amine | 321.00 |
| 1-20 | 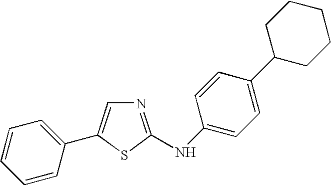 | N-(4-cyclohexylphenyl)-5- phenyl-1,3-thiazol-2-amine | 335.16 |
| 1-21 | 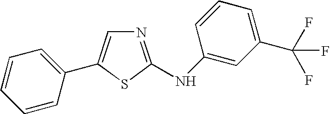 | 5-phenyl-N-[3- (trifluoromethyl)phenyl]- 1,3-thiazol-2-amine | 321.07 |
| 1-22 | 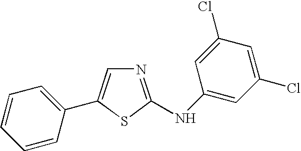 | N-(3,5-dichlorophenyl)-5- phenyl-1,3-thiazol-2-amine | 321.00 |
| 1-23 | 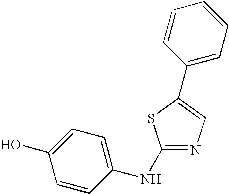 | 4-[(5-phenyl-1,3-thiazol-2- yl)amino]phenol | 268.05 |
| 1-24 | 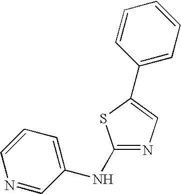 | N-(5-phenyl-1,3-thiazol-2- yl)pyridin-3-amine | 254.1 |
| 1-25 | 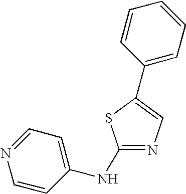 | N-(5-phenyl-1,3-thiazol-2- yl)pyridin-4-amine | 254.1 |

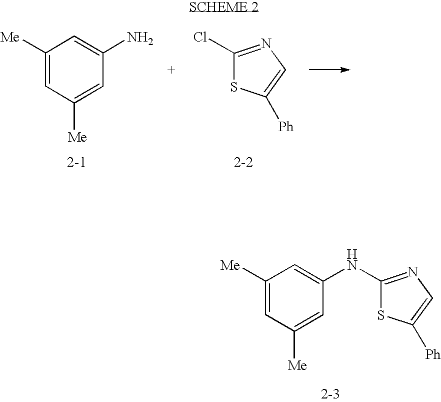 N-(3,5-dimethylphenyl)-5-phenyl-1,3-thiazol-2-amine (2-3)

3,5-Dimethylaniline (2-1, 0.320 mL, 2.56 mmol) and 2-chloro-5-phenyl-1,3-thiazole (2-2, Hafez, E. A. A.; Abed, N. M.; Elsakka, I. A. J. Heterocycl. Chem. 1983; 20, 285-288, 0.100 g, 0.511 mmol) were heated to 100° C. After 1 hour the reaction was warmed to 130° C. After 1 hour at this temperature the reaction was cooled to room temperature. The reaction was diluted with 1 M HCl (aq) and extracted 3× with dichloromethane. The combined extracts were dried over Na₂SO₄, filtered and concentrated. The residue was purified by flash column chromatography (dissolving sample in DMF and eluting with 87:13 hexanes/ethyl acetate) to provide pure desired product. ¹H NMR (CDCl₃) δ 7.44 (bm, 6H), 7.01 (s, 2H), 6.93 (s, 1H), 2.36 (s, 6H). MS [M+H]+=281.0. mp=144° C. 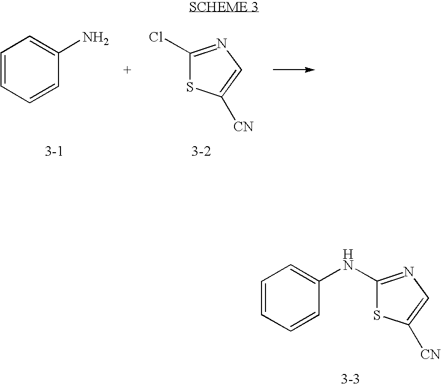
2-Anilino-1,3-thiazole-5-carbonitrile (3-3)

Aniline (3-1, 0.315 mL, 3.46 mmol) and 2-chloro-thiazole-5-carbonitrile (3-2, WO0117995, 0.100 g, 0.692 mmol) were heated to 50° C. After 20 minutes the reaction was warmed to 70° C. After 2 hours at this temperature the reaction was cooled to room temperature and the product was purified by reverse phase preparative chromatography. ¹H NM (DMSO-4) δ 10.99 (s, 1H), 8.13 (s, 1H), 7.60 (d, 2H, J=7.8 Hz), 7.37 (t, 2H, J=7.6 Hz), 7.08 (t, 1H, J=7.3 Hz). MS [M+H]+=202.1. mp=193-194° C. 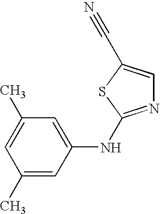
2-[(3.5-dimethylphenyl)amino]-1,3-thiazole-5-carbonitrile (3-4)

3,5-Dimethylaniline (01.431 mL, 3.46 mmol) and 2-chloro-thiazole-5-carbonitrile (3-2, 0.100 g, 0.692 mmol) were heated to 70° C. for 1.5 hours. The reaction was diluted with 1 M HCl (aq) and extracted 3× with dichloromethane. The combined extracts were dried over Na₂SO₄, filtered and concentrated. The residue was purified by flash column chromatography (eluting with 3:1 hexanes/ethyl acetate) to provide pure desired product. ¹H NMR (CDCl₃) δ 7.90 (bs, 1H), 7.77 (s, 1H), 6.93 (s, 2H), 6.87 (s, 1H), 2.35 (s, 6H). MS [M+H]+=230.1. mp=200° C.

The following compounds were made in a similar fashion.

| | | | MS |
|---|---|---|---|
| No. | Structure | Name | (M + H) |
| 3-5 | 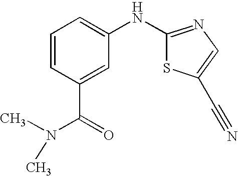 | 3-[(5-cyano-1,3-thiazol-2- yl)amino]-N,N-dimethylbenzamide | 273.0 |
| 3-6 | 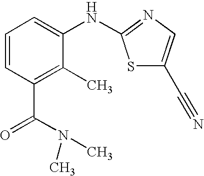 | 3-[(5-cyano-1,3-thiazol-2- yl)amino]-N,N,2-trimethylbenzamide | 287.2 |
| 3-7 | 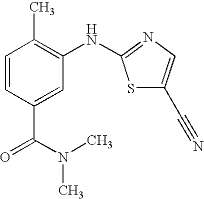 | 3-[(5-cyano-1,3-thiazol-2- yl)amino]-N,N,4-trimethylbenzamide | 287.2 |
| 3-8 | 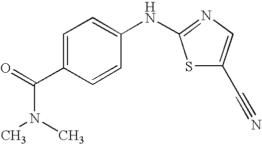 | 4-[(5-cyano-1,3-thiazol-2- yl)amino]-N,N-dimethylbenzamide | 273.2 |
| 3-9 | 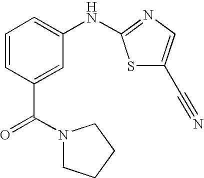 | 2-{[3-(pyrrolidin-1- ylcarbonyl)phenyl]amino}- 1,3-thiazole-5-carbonitrile | 299.2 |
| 3-10 | 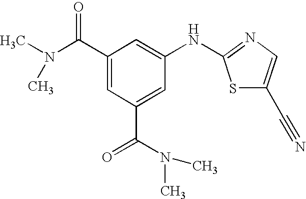 | 5-[(5-cyano-1,3-thiazol-2- yl)amino]-N,N,N′,N′- tetramethyl isophthalamide | 344.2 |
| 3-11 | 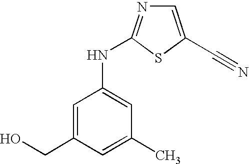 | 2-{[3-(hydroxymethyl)-5- methylphenyl]amino}-1,3- thiazole-5-carbonitrile | 246.2 |

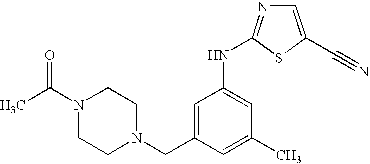

2-({3-[(4-Acetylpiperazin-1-yl)methyl]-5-methylphenyl}amino)-1,3-thiazole-5-carbonitrile (3-12)

2-{[3-(Hydroxymethyl)-5-methylphenyl]amino}-1,3-thiazole-5-carbonitrile (3-11, 0.030 g, 0.122 mmol) was dissolved in 1 ml anhydrous dichloromethane. Phosphorous oxychloride (0.011 mL, 0.12 mmol) was addded followed by addition of anhydrous dimethylformamide (0.009 mL, 0.12 mmol). The reaction was stirred at ambient temperature for 18 hours. The reaction was quenched with water and extracted 2× with dichloromethane. The organic extracts were combined, dried over Na₂SO₄, filtered and concentrated. To the residue from this reaction was added 1-acetylpiperazine (0.063 g, 0.49 mmol) and 1 ml dimethylsulfoxide. The resulting solution was heated to 70° C. for 1 hour. The reaction was directly purified by reverse phase preparative HPLC to provide the desired product as its trifluoroacetate salt. ¹H N (CDCl₃) δ 7.78 (s, 1H), 7.44 (s, 2H), 6.93 (s, 1H), 4.11 (s, 2H), 3.82 (m, 2H), 3.10 (bs, 6H), 2.39 (s, 3H), 2.12 (s, 3H). MS [M+H]+=356.3. 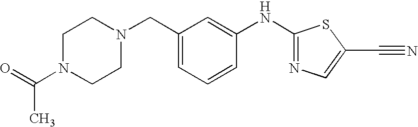
2-({3-[(4-Acetylpiperazin-1-yl)methyl]phenyl}amino)-1,3-thiazole-5-carbonitrile (3-13)

3-Nitrobenzaldehyde (0.384 g, 2.54 mmol) was dissolved in 10 mL of 1,2-dichloroethane. 1-Acetylpiperazine (0.358 g, 2.80 mmol) was added followed by the addition of sodium triacetoxyborohydride (0.592 g, 2.80 mmol) and acetic acid (0.200 mL, 3.49 mmol). After 16 hours the reaction was diluted with saturated aqueous NaHCO₃. The resulting mixture was extracted 2× with dichloromethane and the combined organic extracts were dried over Na₂SO₄, filtered and concentrated. Purification of the residue by flash column chromatography provided pure 1-acetyl-4-(3-nitrobenzyl)piperazine. 1-Acetyl-4-(3-nitrobenzyl)piperazine (0.387 g, 1.47 mmol) was dissolved in 5 mL EtOH. Tin(II) chloride dihydrate (1.66 g, 7.35 mmol) was added and the resulting mixture was heated to reflux. After 3 hours the reaction was cooled and quenched with water. The mixture was extracted 3× with dichloromethane and the combined organic extracts were dried over Na₂SO₄, filtered and concentrated to afford reasonably pure 3-[(4-acetylpiperazin-1-yl)methyllaniline. 3-[(4-Acetylpiperazin-1-yl)methyl]aniline (0.089 g, 0.381 mmol) and 2-chloro-thiazole-5-carbonitrile (3-2, 0.061 g, 0.42 mmol) were mixed and heated to 80° C. for 1 hour. The reaction was cooled and purified directly by reverse phase preparative HPLC to afford a pure sample of the title product. ¹H NMR (CDCl₃) δ 9.41 (bs, 1H), 7.80 (s, 1H), 7.32-7.40 (m, 3H), 7.13 (d, 1H, J=7.1 Hz), 3.65 (t, 2H, J=4.8 Hz), 3.56 (s, 2H), 3.50 (t, 2H, J=4.9 Hz), 2.49 (t, 2H, J=5.0 Hz), 2.44 (t, 2H, 5.0 Hz), 2.12 (s, 3H). MS [M+H]+=342.08. 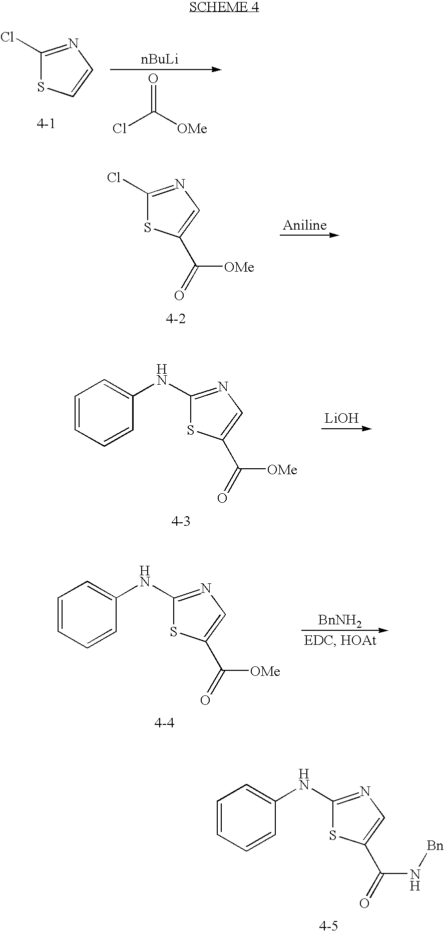
Methyl 2-anilino-1,3-thiazole-5-carboxylate (4-3)

2-Chlorothiazole (0.670 g, 5.60 mmol) was dissolved in 10 mL of anhydrous tetrahydrofuran under Ar and the resulting solution was cooled to −78° C. n-Butyllithium (2.5 M in hexane, 2.46 mL, 6.16 mmol) was added dropwise. After 15 minutes, methyl chloroformate (0.650 mL, 8.40 mmol) was added. After 30 minutes the reaction was allowed to warm to ambient temperature and was quenched by the addition of half-saturated aqueous NHCl. The resulting mixture was extracted 3× with dichloromethane and the organic extracts were dried over Na₂SO₄, filtered and concentrated. ¹H NMR showed pure methyl 2-chloro-1,3-thiazole-5-carboxylate (4-2). 2-Chloro-1,3-thiazole-5-carboxylate (4-2, 0.713 g, 4.01 mmol) was stirred with aniline (1.83 mL, 20.1 mmol) and the mixture was heated to 100° C. After 4 hours the reaction was cooled, diluted with ethyl acetate and washed with 0.5 M aqueous HCl. The aqueous phase was extracted 2× with ethyl acetate and the combined organic extracts were dried over Na₂SO₄, filtered and concentrated. The residue was purified by flash column chromatography to afford the title compound. ¹H NMR (CDCl₃) δ 7.96 (s, 1H), 7.69 (bs, 1H), 7.41 (t, 2H, J=8.6 Hz), 7.35 (d, 2H, J=8.8 Hz), 7.17 (t, 1H, J=7.2 Hz), 3.85 (s, 3H). MS [M+H]+=235.0. mp=175-176° C.

2-Anilino-1,3-thiazole-5-carboxylic Acid (4-4)

Methyl 2-anilino-1,3-thiazole-5-carboxylate (4-3, 0.200 g, 0.854 mmol) was dissolved in 4 mL of 3:1 MeOH/water. Lithium hydroxide hydrate (0.179 g, 4.27 mmol) was added and the reaction was stirred at ambient temperature for 1 hour. The mixture was heated to 50° C. for 2 hours and was then cooled and diluted with water. The solution was adjusted to pH 4 with 1M aqueous HCl. A precipitate formed which was filtered and washed with water. ¹H NMR (DMSO-4) δ 12.8 (bs, 1H), 10.7 (s, 1H), 7.6 (d, 2H), 7.4 (t, 2H), 7.0 (t, 1H). MS [M+H]+=221.1.

2-Anilino-N-benzyl-1.3-thiazole-5-carboxamide (4-5)

2-Anilino-1,3-thiazole-5-carboxylic acid (4-4, 0.025 g, 0.11 mmol), 1-(3-dimethylaminopropyl)₃-ethylcarbodiimide hydrochloride (0.024 g, 0.13 mmol), 1-hydroxy-7-azabenzotriazole (0.016 g, 0.11 mol) and benzylamine (0.014 mL, 0.13 mmol) were dissolved in 1 mL dimethylformamide. After 3 hours the reaction was purified by reverse phase preparative HPLC followed by flash column chromatography to afford the pure title compound. ¹H NMR (CD₃OD) δ 7.81 (s, 1H), 7.53 (d, 2H, J=8.9 Hz), 7.35-7.22 (m, 7H), 7.04 (t, 1H, J=7.3 Hz), 4.50 (s, 2H). MS [M+H]+=310.0. 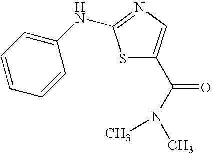
2-Anilino-N,N-dimethyl-1,3-thiazole-5-carboxamide (4-6)

2-Anilino-1,3-thiazole-5-carboxylic acid (4-4, 0.025 g, 0.11 mmol), 1-(3-dimethylaminopropyl)₃-ethylcarbodiimide hydrochloride (0.024 g, 0.13 mmol), 1-hydroxy-7-azabenzotriazole (0.016 g, 0.11 mol), dimethylamine hydrochloride (0.010 g, 0.10 mmol) and N,N-diisopropylethylamine (0.016 g, 0.13 mmol) were dissolved in 1 mL dimethylformamide. The reaction was stirred overnight and was then concentrated in vacuo. The residue was purified by flash column chromatography (gradient elution using 50% to 80% ethyl acetate in dichloromethane) to afford the pure title compound. ¹H NMR (CDCl₃) δ 7.84 (bs, 1H), 7.58 (s, 1H), 7.43-7.34 (m, 4H), 7.17-7.10 (m, 1H), 3.2 (s, 6H). MS [M+H]+=248.1. 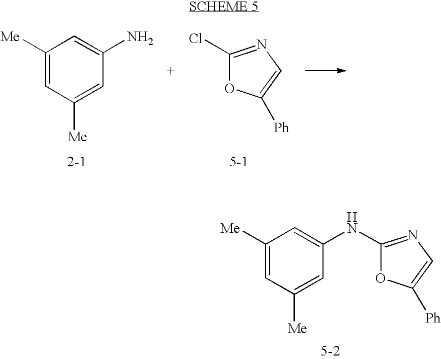
N-(3,5-Dimethylphenyl)-5-phenyl-1,3-oxazol-2-amine (5-2)

A mixture of 2-chloro-5-phenyl-1,3-oxazole (5-1, Vedejs, E.; Monahan, S. D. J. Org. Chem. 1996; 61, 5192-5193, 0.050 g, 0.28 mmol) and 3,5-dimethylaniline (2-1, 0.168 g, 1.39 mmol) was heated to 70° C. After 3 hours the reaction was cooled and diluted with dimethylsulfoxide. The resulting solution was purified sequentially by reverse phase preparative HPLC and flash column chromatography (eluting with 9:1 hexanes/ethyl acetate) to afford pure 5-2. ¹H NMR (CDCl₃) δ 7.54 (d, 2H, J=7.3 Hz), 7.39 (t, 2H, J=7.3 Hz), 7.28-7.25 (m, overlapping with CHCl₃), 7.14 (d overlapping with s, 2H, J=8.3 Hz), 6.80 (bs, 1H), 6.71 (s, 1H), 2.34 (s, 6H). MS [M+H]+=265.1. mp=194° C. 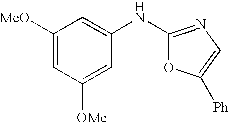
N-(3.5-Dimethoxyphenyl)-5-phenyl-1.3-oxazol-2-amine (5-3)

A mixture of 2-chloro-5-phenyl-1,3-oxazole (5-1, 0.050 g, 0.28 mmol) and 3,5-dimethoxyaniline (2-1, 0.213 g, 1.39 mmol) was heated to 70° C. After 3 hours the reaction was cooled and diluted with dimethylsulfoxide. The resulting solution was purified sequentially by reverse phase preparative HPLC and flash column chromatography (eluting with 82:18 hexanes/ethyl acetate) to afford pure 5-2. ¹H NMR (CDCl₃) δ 7.54 (d, 2H, J=7.0 Hz), 7.39 (t, 2H, J=7.3 Hz), 7.29-7.25 (m, overlapping with CHCl₃), 7.16 (s overlapping with bs, 2H), 6.74 (d, 2H, J=2.1 Hz), 6.19 (tm 1H, J=2.2 Hz), 3.83 (s, 6H). MS [M+H]+=297.1. mp=173° C.

The following compounds were made in a similar fashion.

| | | | MS | |
|---|---|---|---|---|
| No. | Structure | Name | (M + H) | mp |
| 5-4 | 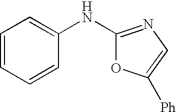 | N,5-diphenyl-1,3-oxazol-2-amine | 237.1 | 169-170° C. |
| 5-5 | 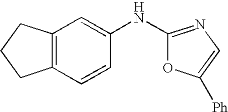 | N-(2,3-dihydro-1H-inden-5-yl)- 5-phenyl-1,3-oxazol-2-amine | 277.1 | 152° C. |
| 5-6 | 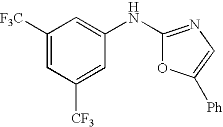 | N-[3,5-bis(trifluoromethyl)- phenyl]-5-phenyl-1,3-oxazol-2-amine | 372.9 | 203° C. |

1. A compound of Formula I: 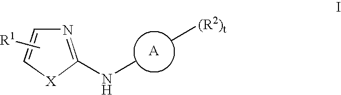
wherein 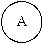
is an aryl or heterocyclyl;
X is S or O;
R is independently selected from
1) H,
2) unsubstituted or substituted C₁-C₁₀ alkyl,
3) (CR₂)ₙOR⁴, and
4) unsubstituted or substituted aryl;
R¹ is
1) unsubstituted or substituted phenyl,
2) CN, or
3)—C(O)NR42;
R² is independently selected from:
1) H,
2) CN,
3) Halo,
4) (CR₂)ₙOR⁴,
5) CF₃,
6) unsubstituted or substituted C₁-C₁₀ alkyl,
7) unsubstituted or substituted C₃-C₁₀ cycloalkyl,
8) unsubstituted or substituted aryl,
9) unsubstituted or substituted aralkyl,
10) unsubstituted or substituted heterocyclyl,
11) unsubstituted or substituted heterocyclylalkyl,
12)—CR₂)ₙC(O)NR⁴₂,
13)—CR₂)ₙC(O)R⁴,
14)—CR₂)ₙR⁶C(O)R⁴₂, and
15)—CR₂)ₙC(O)OR⁴;
R⁴ is independently selected from:
1) H, 2) unsubstituted or substituted C₁-C₁₀ alkyl,
3) unsubstituted or substituted aryl,
4) unsubstituted or substituted aralkyl,
5) unsubstituted or substituted heterocyclyl, and
6) unsubstituted or substituted heterocyclylalkyl;
R⁶ is unsubstituted or substituted heterocyclyl;
n is independently 0, 1, 2, 3, 4, 5, or 6;
t is 0, 1, 2, or 3;
or a pharmaceutically acceptable salt thereof.

2. The compound of claim 1 wherein 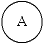
is an aryl or pyridyl;
n is independently 0, 1, 2, 3, or 4;
or a pharmaceutically acceptable salt thereof.

3. The compound of claim 1 wherein 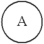
is phenyl, naphthyl, dihydroindenyl, indenyl, or pyridyl;
R is independently selected from
1) H,
2) unsubstituted or substituted C₁-C₁₀ alkyl, and
3) (CR₂)ₙOR⁴;
R² is independently selected from:
1) H,
2) CN,
3) Halo,
4) (CR₂)ₙOR⁴,
5) CF₃,
6) unsubstituted or substituted C₁-C₁₀ alkyl,
7) unsubstituted or substituted C₃-C₁₀ cycloalkyl,
8)—CR₂)ₙC(O)NR⁴₂,
9) —(CR₂)ₙC(O)R⁴, and
10)—CR₂)ₙR⁶C(O)R⁴₂;
n is independently 0, 1, 2, 3, or 4;
or a pharmaceutically acceptable salt thereof.

4. A compound selected from:
N,5-diphenyl-1,3-thiazol-2-amine;
N-(1-naphthyl)-5-phenyl-1,3-thiazol-2-amine;
N-(3-methoxyphenyl)-5-phenyl-1,3-thiazol-2-amine;
N-[4-(benzyloxy)phenyl]-5-phenyl-1,3-thiazol-2-amine;
N-(4-methoxyphenyl)-5-phenyl-1,3-thiazol-2-amine;
4-[(5-phenyl-1,3-thiazol-2-yl)amino]benzonitrile;
N-(2-chlorophenyl)-5-phenyl-1,3-thiazol-2-amine;
N-(2,4-dimethoxyphenyl)-5-phenyl-1,3-thiazol-2-amine;
N-(3-chlorophenyl)-5-phenyl-1,3-thiazol-2-amine;
N-(4-phenoxyphenyl)-5-phenyl-1,3-thiazol-2-amine;
N-(2,5-dimethoxyphenyl)-5-phenyl-1,3-thiazol-2-amine;
N-(2,5-dichlorophenyl)-5-phenyl-1,3-thiazol-2-amine;
N-(4-chlorophenyl)-5-phenyl-1,3-thiazol-2-amine;
N-(2,6-dichlorophenyl)-5-phenyl-1,3-thiazol-2-amine;
N-(2-methoxyphenyl)-5-phenyl-1,3-thiazol-2-amine;
N-(2,4-dichlorophenyl)-5-phenyl-1,3-thiazol-2-amine;
N-(3,4-dichlorophenyl)-5-phenyl-1,3-thiazol-2-amine;
N-(4-cyclohexylphenyl)-5-phenyl-1,3-thiazol-2-amine;
5-phenyl-N-[3-(trifluoromethyl)phenyl]-1,3-thiazol-2-amine;
N-(3,5-dichlorophenyl)-5-phenyl-1,3-thiazol-2-amine;
4-[(5-phenyl-1,3-thiazol-2-yl)amino]phenol;
N-(3,5-dimethylphenyl)-5-phenyl-1,3-thiazol-2-amine;
2-Anilino-1,3-thiazole-5-carbonitrile;
2-[(3,5-dimethylphenyl)amino]-1,3-thiazole-5-carbonitrile;
3-[(5-cyano-1,3-thiazol-2-yl)amino]-N,N-dimethylbenzamide;
3-[(5-cyano-1,3-thiazol-2-yl)amino]-N,N,2-trimethylbenzamide;
3-[(5-cyano-1,3-thiazol-2-yl)amino]-N,N,4-trimethylbenzamide;
4-[(5-cyano-1,3-thiazol-2-yl)amino]-N,N-dimethylbenzamide;
2-{[3-(pyrrolidin-1-ylcarbonyl)phenyl]amino}-1,3-thiazole-5-carbonitrile;
5-[(5-cyano-1,3-thiazol-2-yl)amino]-N,N,N′,N-tetramethylisophthalamide;
2-{[3-(hydroxymethyl)-5-methylphenyl]amino}-1,3-thiazole-5-carbonitrile;
2-({3-[(4-Acetylpiperazin-1-yl)methyl]-5-methylphenyl}amino)-1,3-thiazole-5-carbonitrile;
2-({3-[(4-Acetylpiperazin-1-yl)methyl]phenyl}amino)-1,3-thiazole-5-carbonitrile;
Methyl 2-anilino-1,3-thiazole-5-carboxylate;
2-Anilino-1,3-thiazole-5-carboxylic acid;
2-Anilino-N-benzyl-1,3-thiazole-5-carboxamide;
2-Anilino-N,N-dimethyl-1,3-thiazole-5-carboxamide;
N-(3,5-Dimethylphenyl)-5-phenyl-1,3-oxazol-2-amine;
N-(3,5-Dimethoxyphenyl)-5-phenyl-1,3-oxazol-2-amine;
N,5-diphenyl-1,3-oxazol-2-amine;
N-(2,3-dihydro-1H-inden-5-yl)-5-phenyl-1,3-oxazol-2-amine;
N-[3,5-bis(trifluoromethyl)phenyl]-5-phenyl-1,3-oxazol-2-amine;
N-(5-phenyl-1,3-thiazol-2-yl)pyridin-3-amine;
N-(5-phenyl-1,3-thiazol-2-yl)pyridin-4-amine;
or a pharmaceutically acceptable salt thereof.

5. The compound of claim 4 which is selected from
N-(3,5-dimethylphenyl)-5-phenyl-1,3-thiazol-2-amine;
2-[(3,5-dimethylphenyl)amino]-1,3-thiazole-5-carbonitrile; or
2-({3-[(4-Acetylpiperazin-1-yl)methyl]-5-methylphenyl}amino)-1,3-thiazole-5-carbonitrile;
or a pharmaceutically acceptable salt thereof.

6. A pharmaceutical composition which is comprised of a compound in accordance with claim 1 and a pharmaceutically acceptable carrier.

7. A method of treating or preventing cancer in a mammal in need of such treatment which is comprised of administering to said mammal a therapeutically effective amount of a compound of claim 1.

8. A method of treating cancer or preventing cancer in accordance with claim 7 wherein the cancer is selected from cancers of the brain, genitourinary tract, lymphatic system, stomach, larynx and lung.

9. A method of treating or preventing cancer in accordance with claim 7 wherein the cancer is selected from histiocytic lymphoma, lung adenocarcinoma, small cell lung cancers, pancreatic cancer, gioblastomas and breast carcinoma.

10. A method of treating or preventing a disease in which angiogenesis is implicated, which is comprised of administering to a mammal in need of such treatment a therapeutically effective amount of a compound of claim 1.

11. A method in accordance with claim 10 wherein the disease is an ocular disease.

12. A method of treating or preventing retinal vascularization which is comprised of administering to a mammal in need of such treatment a therapeutically effective amount of compound of claim 1.

13. A method of treating or preventing diabetic retinopathy which is comprised of administering to a mammal in need of such treatment a therapeutically effective amount of compound of claim 1.

14. A method of treating or preventing age-related macular degeneration which is comprised of administering to a mammal in need of such treatment a therapeutically effective amount of a compound of claim 1.

15. The composition of claim 6 further comprising a second compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HW protease inhibitor,
9) a reverse transcriptase inhibitor,
10) an angiogenesis inhibitor,
11) a PPAR-γ agonists,
12) a PPAR-δ agonists,
13) an inhibitor of inherent multidrug resistance,
14) an anti-emetic agent,
15) an agent useful in the treatment of anemia,
16) an agent useful in the treatment of neutropenia,
17) an immunologic-enhancing drug,
18) an inhibitor of cell proliferation and survival signaling, and
19) an agent that interferes with a cell cycle checkpoint.

16. A method of treating cancer which comprises administering a therapeutically effective amount of a compound of claim 1 in combination with radiation therapy.

17. A method of treating or preventing cancer which comprises administering a therapeutically effective amount of a compound of claim 1 and paclitaxel or trastuzumab.

18. A method of treating or preventing cancer which comprises administering a therapeutically effective amount of a compound of claim 1 and a GPIIb/IIIa antagonist.

19. The method of claim 18 wherein the GPIIb/IIIa antagonist is tirofiban.

20. A method of treating or preventing diabetic retinopathy which comprises administering a therapeutically effective amount of a compound of claim 1 in combination with a PPAR-γ agonist.
